SEQUENCE LISTING (14 sequences)

SEQ ID NO: 1 - DNA, 27 bp, Artificial Sequence
  Description of Artificial Sequence primer1
ggaattcgccaccatggtgagcaaggg

SEQ ID NO: 2 - DNA, 31 bp, Artificial Sequence
  Description of Artificial SequencePrimer 2
gaagatctttacttgtacagctcgtccatgc

SEQ ID NO: 3 - DNA, 31 bp, Artificial Sequence
  Description of Artificial SequencePrimer 3
ccgctcgagcgcataagagtcccaaagctcc

SEQ ID NO: 4 - DNA, 31 bp, Artificial Sequence
  Description of Artificial SequencePrimer 4
cccaagcttgggtactttctgccgaaatgag

SEQ ID NO: 5 - DNA, 33 bp, Artificial Sequence
  Description of Artificial Sequence Primer 5
ggaattccatggcacaggttatcaacacgtttg

SEQ ID NO: 6 - DNA, 30 bp, Artificial Sequence
  Description of Artificial Sequence Primer 6
gaagatctttatctgatttttgtaaaggtc

SEQ ID NO: 7 - DNA, 27 bp, Artificial Sequence
  Description of Artificial Sequence Primer 7
cggaattccacatgaaaaaagcagtca

SEQ ID NO: 8 - DNA, 28 bp, Artificial Sequence
  Description of Artificial SequencePrimer 8
cgggatcccgtattaagaaagtatgatg

SEQ ID NO: 9 - DNA, 16 bp, Artificial Sequence
  Description of Artificial SequencePrimer 9
taggcgtgtacggtgg

SEQ ID NO: 10 - DNA, 20 bp, Artificial Sequence
  Description of Artificial Sequence Primer10
taccacatttgtagaggttt

SEQ ID NO: 11 - DNA, 33 bp, Artificial Sequence
  Description of Artificial SequencePrimer 11
gctctagagcatggcacaggttatcaacacgtt

SEQ ID NO: 12 - DNA, 30 bp, Artificial Sequence
  Description of Artificial SequencePrimer 12
gctctagacgttatctgatttttgtaaagg

SEQ ID NO: 13 - DNA, 22 bp, Artificial Sequence
  Description of Artificial SequencePrimer 13
ccgctcgagtaggcgtgtacgg

SEQ ID NO: 14 - DNA, 29 bp, Artificial Sequence
  Description of Artificial SequencePrimer 14
tccccgcggttacaatttggactttccgc

(19) United States
(12) Patent Application Publication
(10) Pub. No.: US 2004/0034879 A1
(43) Pub. Date: Feb. 19, 2004

(54) Mammalian sex selection using genetic modification

(76) Inventors: Steve Rothstein, Guelph (CA); Alan Wildeman, Guelph (CA)

Correspondence Address: MORGAN & FINNEGAN, L.L.P., 345 PARK AVENUE, NEW YORK, NY, 10154, US

(21) Appl. No.: 10/416,417

(22) PCT Filed: Nov. 9, 2001
(86) PCT No.: PCT/CA01/01605

(30) Foreign Application Priority Data
Nov. 9, 2000 (US) 09/708,734

(51) Int. Cl. A01K 67/00; A61K 48/00; A01K 31/19; C12N 15/86; A01K 67/027

(52) U.S. Cl. 800/8; 435/456; 424/93.2; 119/300

(57) ABSTRACT

A method of sex selection in non-human animals is disclosed which comprises transgenic manipulation of the male to modify, destroy or identify gametes having one of either the X or Y chromosome. A chimeric construct comprising a post-meiotic regulatory region in operative association with a sex selection gene is targeted to one of either a Y or X chromosome in the genome of a male animal. The post-meiotic regulatory region may be transition protein 1 promoter. The selection sequence may be a direct or indirect mediator, or a detectable marker. An example of a direct mediator is the Rnase, Barnase. An example of an indirect mediator is the herpes simplex virus thymidine kinase gene, which forms a toxic metabolite in the presence of gancyclovir. Alternatively, a transcriptional activator protein may be linked to the post-meiotic regulatory element and the direct or indirect mediator may be linked to a promoter having a binding site specific for the transcriptional activator. According to the invention a non-human animal is formed which is capable of producing either exclusively male or exclusively female offspring, or a non-human animal is obtained from which X or Y containing gametes may be easily sorted and isolated. The invention is useful in applications which would benefit from the ability to obtain animals of a desired sex.

This application claims the benefit of priority of U.S. application Ser. No. 09/708,734, filed Nov. 9, 200, which claims priority from 06/64,333, filed Nov. 9, 1999.

The present invention relates to a method of transgenic manipulation to effect sex selection in mammals, and also relates to a genetically modified mammal. More specifically, this invention relates to the selective production of either a male or a female animal.

### BACKGROUND OF THE INVENTION

In mammals, males possess an X and a Y chromosome, whereas females possess two X chromosomes. The sex of an offspring is determined by whether a haploid sperm cell carries an X or a Y chromosome, which, like the 22 pairs of autosomes, segregate during meiosis.

There are strikingly different roles for male and female animals in agriculture, particularly with cattle. Dairy production requires female animals, while beef production relies mainly on males. In other agricultural species such as pigs, sheep and goats, there are also market advantages associated with males or females.

Because of the agricultural implications of the sex of animals, there have been many efforts made over the years to devise strategies for pre-selecting the sex of an offspring, to avoid the time and costs associated with full gestation and rearing of an animal that may not be of the desired sex. Conventional sex pre-selection strategies require technical or veterinary intervention, and frequently employ some form of manipulation of the gamete or embryo, which increases the cost to the producer to a level that prevents widespread application.

Conventional strategies to effect sex selection at various stages from gamete to fetus have met with varying degrees of success. At the gamete stage, attempts have been made to separate X-bearing sperm from Y-bearing sperm, so that fertilization, either in vitro or through artificial insemination, can then be carried out with the likelihood of the desired sex arising. Johnson (J. Reprod Fertil. Suppl; 1997;52:255-266) reports a method incorporating physical separation of male and female sperm by cell sorting.

At the embryo stage, various technologies have attempted to determine the sex of early cleavage stage embryos prior to committing an animal to a pregnancy. Ellis et al. (Theriogenology; 1988;29:242) report a PCR-based assay for detecting the presence of the Y-chromosome. However, the procedure requires an invasive biopsy of several embryo cells.

U.S. Pat. No. 5,596,089 (Silversides et al.), corresponding to Canadian Patent Application No. 2,142,137, teaches the use of transgenic technology to determine the sex of an offspring. The diphtheria toxin A gene is used to genetically ablate gonadal tissue, specifically the primordial germ cells in a developing embryo. The expression of the toxin gene is then brought under the control of the SRY promoter, which is active in the developing gonad. An insert is included to inactivate the toxin gene, and it is flanked by LOX recombination sites. When transgenic animals carrying this toxin gene are mated to transgenic animals carrying the gene for the CRE recombinase, also under control of the SRY promoter, the embryos produced fail to develop gonadal tissue because the CRE recombinase excises the inactivating insert from the toxin gene and thereby enable it to function again. Thus, this approach requires two lines of transgenic animals.

According to the method disclosed in U.S. Pat. No. 5,596,089, exclusively female phenotype animals can be produced having either XX and XY genotype by ablation of Y-containing gonadal tissue. However, the female progeny produced as a result of the disclosed procedure are sterile, and the procedure does not allow formation of a transgenic animal that produces exclusively male offspring.

At the fetus stage, ultrasound monitoring can be used with reasonable success to determine the sex of the developing animal. However, it can only be done after pregnancy is well established, and termination of the pregnancy at this stage is a complex procedure.

An object of the invention is to provide a method of sex selection which obviates or mitigates one or more of the above-noted limitations of the prior art.

The above object is met by the combination of features of the main claim, the sub-claims disclose further advantageous embodiments of the invention.

### SUMMARY OF THE INVENTION

The invention involves transgenic manipulation of mammalian spermatogenesis to effect sex selection.

According to the invention, there is provided a method for sex selection comprising, introducing a post-meiotically expressed sex selection gene into one of either a Y or X chromosome of a male non-human animal, and propagating the animal.

The present invention is also directed to a method for sex selection comprising the steps of introducing in a male animal a chimeric construct comprising a regulatory region that is active post-meiotically and in operative association with a sex selection gene, wherein both of the regulatory region and the sex selection gene are bound by nucleotide sequences that target the chimeric construct to one of either a Y or X chromosome, and propagating the non-human animal. Preferably the post-meiotic regulatory element, used in the step of introducing, is a transition protein 1 (TP1) regulatory region. Furthermore, the sex selection gene, used in the step of introducing, encodes a direct mediator, an indirect mediator or a marker. Preferably, the direct mediator comprises a ribonuclease, the indirect mediator is HSV tk, and the marker is GFP. The nucleotide sequences that target the chimeric construct comprise sequences of homology within the X chromosome flanking the HPRT locus, or comprise, sequences of homology within the Y chromosome flanking the SRY locus.

This invention is also directed to a method as defined above wherein the sex selection gene, used in the step of introducing, is in operative association with an inducible regulatory element within a first chimeric construct, and a gene of interest encoding a regulatory protein capable of activating the inducible regulatory element is within a second chimeric construct, wherein a post-meiotic promoter sequence is in operative association with the first, second or both the first and second chimeric construct, and wherein the first, second or both first and second chimeric sequence are targeted to either the X or Y chromosome. If the inducible regulatory element comprises a GAL4 upstream activating sequence, then the regulatory protein is a GAL4 transcription activator protein. If the inducible regulatory element comprises a Tet-responsive element, then the regulatory protein is a tet-transcription activator protein.

The present invention also pertans to a method as defined above wherein the sex selection gene, used in the step of introducing, is in operative association with an inducible regulatory element, and is introduced into a first non-human animal, and the method further comprises a second introducing step, comprising introducing into a second non-human animal the post-meiotic promoter sequence in operative association with a gene of interest encoding a regulatory protein capable of activating the inducible regulatory element, and mating the first and second non-human animals to produce progeny.

This invention is also directed to a chimeric construct comprising a post-meiotically active regulatory region in operative association with a sex selection gene, wherein the regulatory region, the sex selection gene, or both the regulatory region, and the sex selection gene are bound by nucleotide sequences that target the chimeric construct to one of either a Y or X chromosome.

Furthermore, this invention is directed to a pair of chimeric constructs comprising a first and a second chimeric construct, the first chimeric construct comprising:

a first regulatory region in operative association with a gene of interest encoding a regulatory protein,

the second construct comprising:

a second regulatory region and an inducible regulatory element capable of regulating the activity of said regulatory region in the presence of said regulatory protein, in operative association with a sex selection gene,

wherein the first, the second, or both the first and the second chimeric constructs are bound by nucleotide sequences that target the first, the second, or independently both the first and the second construct, to one of either a Y or X chromosome; and wherein the first, the second, or both the first and the second regulatory region is a post-meiotically active regulatory region.

This invention also pertains to a transgenic non-human male animal comprising the chimeric construct, the first chimeric construct, the second chimeric construct, or the pair of chimeric consructs as defined above, and progeny of the transgenic male non-human animal.

The present invention also embraces a method of introducing a direct mediator into a host organism comprising, introducing at least one chimeric construct comprising:

i) an inducible, temporal, or cell specific regulatory region in operative association with a direct mediator; and

ii) an additional regulatory region exhibiting minimal activity and in operative association with an inhibitor, wherein the inhibitor is specific for the direct mediator, and

propagating the host animal.

Advantageously, the methods of the present invention result the production of a fertile transgenic mammal that is selectively male or female. Thus, if the progeny are deemed to have desirable traits, the parent may be further propagated or the progeny of the transgenic mammal according to the invention can be propagated normally. Additionally, using the method of the invention requires minimal additional technical intervention once a transgenic animal is established in order to continue production of offspring of a desired sex.

This summary of the invention does not necessarily describe all necessary features of the invention but that the invention may also reside in a sub-combination of the described features.

### BRIEF DESCRIPTION OF THE DRAWINGS

These and other features of the invention will become more apparent from the following description in which reference is made to the appended drawings wherein:

FIG. 1 shows maps of plasmid used in Example 5. FIGS. 1(A) shows pTRE-Barstar, FIG. 1(B) shows pBS-Barstar, FIG. 1(C) shows pBS-Barstar-TRE-PolyA, FIG. 1(D) shows pBS-Barsar-Barnase, FIG. 1(E) shows pBS-Tet-Off-Barstar-Barnase.

FIG. 2 shows luciferase activity in human cells transfected with 0.5 μg of pCMV-Luc and 1 μg of control plasmid DNA (con), with 0.5 g of pCMV-Luc and 1 μg DNA of pBS-Barnase-Barstar (B) or with 0.5 μg of pCMV-Luc and 1 μg DNA of pBS-CMV-tTA-Barnase-Barstar (tTA-B).

FIG. 3 shows the increase in luciferase activity in the presence or absence of tetracycline, after 24 hr (B24) or 48 hr (B48) post transfection. For B24 and B48, transfection was with 1 μg DNA of pBS-CMV-tTA-Barnase-Barstar and 0.5 μg of pCMV-Luc. 1 μg of empty pTRE vector DNA and 0.5 μg of pCMV-Luc were used in the control samples (24 and 48). The luciferase activity in the presence of tetracycline was expressed as a ratio of the activity in the absence of tetracycline.

FIG. 4 shows a schematic map of a targeting vector employed in an aspect of an embodiment of the present invention (Ptp 1=TP1 promoter; eGFP=enhanced GFP coding sequence; NEO=neomycin resistance expression cassette; Phprt=genomic fragment containing promoter of Hprt).

FIG. 5 shows GFP transcribed in transgenic mouse testis. RNA was extracted from about 8 week old wild type mouse testis (Wt) and two transgenic mice testis (Tg1 and Tg2). 10 μg total RNA was loaded on each lane. GFP, HPRT and TP1 coding regions were used as probes for each blot

FIG. 6 shows GFP detection in transgenic mice testis. FIG. 6A shows proteins were extracted from about 8 week old wild type mouse testis (Wt) and two transgenic mice testis (Tg1 and Tg2). 60 mg protein was loaded on each lane, separated on 12% acrylamide gel, and probed with GFP(FL) primary antibody and subsequently with anti-rabbit IgG-AP secondary antibody. FIG. 6B shows proteins extracted from heart (H), intestines (I), kidney (K), liver (li), lung (lu), muscle (M) and testis (T) of a transgenic mouse. Protein from wild type mouse testis (W) was used as a negative control.

### DESCRIPTION OF PREFERRED EMBODIMENTS

The present invention relates to a method of transgenic manipulation to effect sex selection in mammals, and also relates to a genetically modified mammal. More specifically, this invention relates to the selective production of either a male or a female animal.

The following description is of a preferred embodiment by way of example only and without limitation to the combination of features necessary for carrying the invention into effect.

The invention provides a method for making a transgenic male animal having sperm that when crossed with a desired female, results in offspring of only one sex. The sex of the offspring produced can therefore be selected as desired by mating these transgenic animals. The present invention also provides for an animal in which X and Y containing sperm are easily identified and separated, thereby allowing sex selection in animals, as the sorted sperm may be used for artificial insemination to produce progeny of a desired sex.

By “regulatory region” or “regulatory element” it is meant a portion of nucleic acid typically, but not always, upstream of a gene, which may be comprised of either DNA or RNA, or both DNA and RNA. A regulatory element may be capable of mediating organ specificity, or controlling developmental or temporal gene activation. A “regulatory element” includes promoter elements, basal (core) promoter elements, elements that are inducible in response to an external stimulus, elements that mediate promoter activity such as negative regulatory elements, transcriptional enhancers, or response elements. “Regulatory element”, as used herein, also includes elements that are active following transcription, for example, regulatory elements that modulate gene expression such as translational and transcriptional enhancers, translational and transcriptional repressors, and mRNA instability determinants. Several of these latter elements may be located proximal to the coding region. In the context of this disclosure, the term “regulatory element” or “regulatory region” typically refers to a sequence of DNA, usually, but not always, upstream (5′) to the coding sequence of a structural gene, which controls the expression of the coding region by providing the recognition for RNA polymerase and/or other factors required for trnscription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. A promoter element comprises a basal promoter element, responsible for the initiation of transcription, as well as other regulatory elements (as listed above) that modify gene expression.

Preferably, the regulatory element of the present invention is active in an temporal and organ specific manner, for example, a regulatory element that is active post-meiotically within a sperm cell. An example of such a regulatory element, which is not to be considered limiting in any manner, is the regulatory element obtained from transition protein 1 (TP1; Yelick et al., Genomics, 1991;11:687-694; Kim et al., DNA Seq., 1992;3:123-125).

Histone proteins are important for the packaging of DNA into chromosomes. During post-meiotic maturation of sperm cells, histone proteins are replaced by transition proteins. The gene encoding one of the major transition proteins, transition protein 1, has been cloned from several species, including mouse (Yelick et al., Genomics, 1991;11:687-694) and cow (Kim et al., DNA Seq., 1992;3:123-125). Transition protein 1 (TP1) is expressed post-meiotically, which ensures that at the time of gene expression, the X and Y chromosomes have already segregated into separate spermatocytes. The regulatory region obtained from TP1 therefore directs expression of a gene of interest that is in operative association therewith, post meiotically. It is to be understood that regulatory regions obtained from other genes that they are expressed post-meiotically may also be used for the present invention as describe herein.

One approach to a method for sex selection of the present invention comprises introducing at least one chimeric construct comprising a regulatory element in operative association with a gene of interest, preferably a sex selection gene, and a nucleotide sequence that provides for site specific introduction of the chimeric construct within either the Y or X chromosome, and introducing this chimeric construct into a male animal. In this manner the chimeric construct is specifically targeted to only one of the Y or X chromosomes. The post-meiotic expression of the gene of interest may, but not necessarily, lead to the killing of the cell comprising the chimeric construct.

The gene of interest is preferably a sex selection gene. By “a sex selection gene” it is meant a gene that encodes a protein that is a modifier that either directly or indirectly mediates cellular processes that result in cell death, a protein that interrupts developmental process of the cell required for fertilization, a protein that is a marker, or a combination thereof. However, it is to be understood that the term “gene of interest”, may also refer to genes other than a sex selection gene. For example, which is not to be considered limiting in any manner, a gene of interest may encode a transcriptional activator used to regulate the expression of a sex selection gene.

The expression of the gene of interest, preferably a sex selection gene, may be under the control of a regulatory element that is inducible, there by permitting selective expression of the gene of interest. In this latter embodiment, expression of the gene of interest, preferably a sex selection gene, may directly kill the cell (i.e. a direct mediator), or may be active in killing the cell in the presence of a compound that is metabolized into a toxic compound that eventually kills the cell or alters a process related to fertilization (an indirect mediator), or it may be a marker protein.

The chimeric constructs comprising the sequences of the present invention can be introduced into a male animal by any suitable method, for example which is not to be considered limiting in a any mamner, the transformation of embryonal stem cells (e.g. Robertson, E. J., 1991,. Biol. Reprod. 44:238-45), thereby producing a transgenic animal having the required elements. Preferably the chimeric constructs are introduced, in a site specific manner, into regions of the either the X or Y chromosome that are transcriptionally active, for example but not limited to, in the case of the X chromosome, targeting to the HPRT locus. An example of a Y chromosome target includes, but is not limited to, the SRY locus (Capel, B., 1998, Annu. Rev. Physiol. 60:497-523. ). Methods for site specific integration of the construct within a chromosome are known within the art, for example but not limited to homologous recombination.

The occurrence of the transgene is confirmed within transgenic animals by any suitable method including Southern analysis, PCR or any others suitable method known to one of skill in the art. Such transgenic animals may be used as founder animals and crossed as desired with wild-type or other transgenic animals as discussed further below.

When site specific expression of the gene of interest, preferably a sex selection gene, is not desired, selected chimeric constructs may also be introduced into an animal by DNA microinjection (Gordon, J. W., Scangos, D. J., Plotkin, D. J., Barbosa, J. A. and Ruddle, F. H. 1980, Proc. Natl. Acad. Sci. 77:7380-7384.), or any other method that permits expression of the chimeric construct within the recipient cell.

According to the present invention, the gene of interest is preferably a sex selection gene capable of encoding a protein that either:

1. is a modifier, wherein this modifier either directly or indirectly mediates the development of a cell. A modifier that directly mediates cell development is termed herein a “direct mediator”, while a modifier that indirectly mediates cell development is termed herein, an “indirect mediator”. Collectively, direct, indirect or both direct and indirect mediators are termed herein as a “modifier”. By modifying or mediating the development of a cell, it is meant that the expression of the gene of interest effects cellular process that either results in cellular death, or that results in the cells inability to fertilize an egg cell and produce a zygote. The gene of interest may effect cellular death or alter the development of the sperm cell in either a direct or indirect manner. An example, which is not to be considered limiting in any manner, of a protein produced by a gene of interest that directly mediates the development of a cell includes ribonuclease, for example, but not limited to Barnase. An example, which again is not to be considered limiting in any manner, of a protein that indirectly mediates the development of a cell is an enzyme that metabolizes an innocuous compound to a compound that may kill the cell, for example, but not limited to, thymidine kinase gene from herpes simplex virus (HSV tk) which encodes a product that metabolises gancyclovir to metabolites that are toxic to the cell. An example of a protein that results in the inability of a cell expressing this protein to fertilize a zygote includes, but is not limited to, cell surface receptor proteins involved in egg cell recognition. However, it is to be understood that these are examples only, and that other proteins may also be used for these purposes;

2. is used as a marker of a transformed cell for example but not limited to GFP (Green Fluorescent Protein), GUS, luciferase, CAT, or any other marker that may be suitable for detecting a transformed cell visually, enzymatically or by using cell sorting devices, for example FACS; or

3. is a protein (a regulatory protein) that induces the expression of a second gene of interest, or sex selection gene, in the same or a different chimeric construct by binding or in some manner activating an inducible regulatory element (i.e. a two gene system). An example of this regulatory protein, which is not to be considered limiting in any manner, is a DNA binding protein that regulates the expression of a second gene of interest, typically in trans. The expression of the second gene of interest encodes a protein that modifies the development of the transformed cell, either directly or indirectly (as described in (1) above). Examples of a regulatory proteins, for example a DNA binding protein, and which is not to be considered limiting, include the GAL4 transcriptional activator that binds and activates an Upstream Activating Sequence (UAS) of a second construct, or a tet-transcription activator (tTA) protein, that reversibly binds the Tet-responsive element (Tet-RE, or TRE) associated with a second construct, permitting the expression of a second gene of interest The UAS and TRE are examples of inducible regulatory elements. Another example of a protein that results in the expression of a second gene of interest includes a recombinase protein, that is able to remove a fragment of DNA (a blocker fragment) within the same, or a second gene of interest. The blocker fragment is located within specific nucleotide sequences recognized by the recombinase. Recognition of these specific sequences by the recombinase results in the excision of the blocker fragment from the construct and permits expression of the second gene of interest. An example of a recombinase, which is not to be considered limiting in any manner, is the Cre recombinase, that recognizes specific lox sequences, however, other recombinase systems that are known in the art may also be used.

In one embodiment of the present invention, by targeting a chimeric construct containing a sex selection gene encoding a modifier, for example a direct mediator, such as but not limited to Barnase, to the X chromosome and by ensuring that this gene is expressed in a tissue, and developmentally, specific manner, for example, post-meiotically, only transformed cells containing an X chromosome will be killed while cells containing a Y chromosome remain viable. If sperm from this transgenic animal are crossed with an egg from a wild-type female, only male (XY) offspring are produced.

If the chimeric construct containing a sex selection gene encoding a modifier is targeted to the Y chromosome, and by ensuring expression of this sex selection gene in a tissue, and developmentally, specific manner, for example, post-meiotically, only transformed cells containing a Y chromosome will be effected, while cells containing an X chromosome remain viable. If sperm from this transgenic animal are crossed with an egg from a wild-type female, only female (XX) offspring are produced.

In an alternate embodiment, a modifier, for example a direct mediator, such as but not limited to Barnase, is encoded by a sex selection gene and located on a second chimeric construct. However, it is to be understood that an indirect mediator may also be used as a sex selection gene located on a second chimeric construct in the method described below. The expression of this sex selection gene is regulated by the expression product of a gene of interest located on a first chimeric construct. In this embodiment, which is not to be considered limiting in any manner, the gene of interest may encode the GAL4 transcriptional activator (a regulatory protein), or tTA (tetracycline trnscriptional activator; a regulatory protein, see below) under control of a regulatory element that permits post-meiotic expression, for example the TP1 regulatory element, and the second chimeric construct may comprise a basal promoter and a GAL4 UAS, or a TRE (tetracycline responsive element, see below), that regulates activity of the basal promoter, both in operative association with Barnase. The expression of Barnase only takes place if the GAL4 transcriptional activator, or tTA, encoded by the gene of interest, binds the UAS, or TRE, respectively, and permits expression of Bamase.

For example; which is not to be considered limiting, if the GAL4 transcriptional activator is targeted to the X chromosome and expressed post meiotically, only cells expressing the gene encoding this protein are killed, in the presence of the second chimeric construct. As long the first, the second, or both the first and the second chimeric construct are targeted to a sex chromosome (either the X or Y chromosome) then post meiotic expression of the targeted chimeric construct will result in expression of the direct or indirect mediator resulting in, for example, cell death. There are many combinations for the targeting and post meiotic expression of either the first or second chimeric construct, or the selection of the components occurring within the first or second chimeric construct, that may result in, for example cell death, provided that the expression of the modifier, for example Bamase, is regulated by a UAS. An example of several of these combinations, which are not to be considered limiting include:

the first chimeric construct comprises TP1 in operative association with a UAS or similar responsive element (RE), and modifier, all bound by HPRT; the second chimeric construct comprises a promoter in operative association with a regulatory protein, for example the transcriptional activator GAL4 or tTA, which is incorporated non-specifically within an autosome;

the first chimeric construct comprises a promoter in operative association with a regulatory protein, and the promoter and activator are bound by HPRT sequences; the second chimeric construct comprises TP1 in operative association with a UAS or RE, and modifier, which are incorporated non-specifically within an autosome;

the first chimeric construct comprises TP1 in operative association with a UAS or RE, and a modifier, all bound by HPRT; the second chimeric construct comprises a promoter in operative association with a regulatory protein, for example GAL4 or tTA, all bound by HPRT

the first chimeric construct comprises TP1 in operative association with a regulatory protein, which are incorporated non-specifically within an autosome; the second chimeric construct comprises a promoter in operative association with a UAS or an RE, which are all bound by HPRT sequences.

If either or both of the first or second gene of interest are targeted to the X chromosome, only cells comprising an X chromosome are killed, and Y containing cells remain viable. If sperm from this transgenic animal are crossed with an egg from a wild-type female, only male (XY) offspring are produced. It is to be understood that either, or both the first or second gene of interest may also be targeted to the Y chromosome. In some situations it may be desirable to have both constructs linked on the same chromosome to ensure that both constructs are passed on to the progeny without segregating. This can be accomplished by any suitable means, for example, but not limited to, transforming a host with a vector comprising both constructs, or targeting each construct to the same X or Y chromosome.

The second chimeric construct comprising the sex selection gene may also be administered to an animal transformed with the first chimeric construct comprising the gene of interest via microinjection, as it is not necessary that the second chimeric construct be located on a sex chromosome. Preferably, the first and second chimeric constructs are located within different transgenic animals, and the interaction described above occurs following mating of the two animals. In this manner it is possible to transmit the sex selection gene and the gene of interest to progeny as desired in order to maintain the specific transformed animal lines.

In another embodiment of the present invention, by targeting a construct containing a sex selection gene encoding an indirect mediator, for example but not limited to, HSV tk to the X chromosome and by ensuring that this gene is expressed in a tissue and developmentally specific manner, for example, post-meiotically, only transformed cells containing an X chromosome will be killed in the presence of gancyclovir, while cells containing a Y chromosome remain viable. Gancyclovir may be administered to an animal transformed with a chimeric construct comprising HSV tk, or this compound may be mixed with mature sperm. If sperm from this transgenic animal is crossed with an egg from a wild-type female, only male (XY) offspring are produced. In this embodiment, the transgene can be transmitted to progeny in the absence of gancyclovir. Furthermore, only a single transgene is required to produce an animal that sires progeny of only one sex.

Similarly, if the Y chromosome is targeted with at least one construct, for example which is not to be considered limiting in any manner, that is targeted to the SRY locus, that is capable of expressing a protein that either directly or indirectly mediator the development of a cell, using any of the methods outlined above, so that the post meiotic expression of the direct or indirect mediator is selectively associated with Y chromosome-containing cells, then only cells comprising an X chromosome remain viable. The crossing of such sperm with a wild-type female results in the production of female (XX) offspring.

If a modifier is toxic to the cell, for example but not limited to Barnase, it will be desired that the expression of the modifier is minimized or attenuated in some manner until its expression is desired within a cell. The present invention provides a method for expressing a cytotoxic gene using, for example, but not limited to, the tetracycline (Tet) transactivator system. The Tet system employs two chimeric constructs; one expressing a synthetic transactivator protein (tTA) driven by a regulatory element, and the other being a gene of interest under the control of a Tet operator minimal promoter, for example a tetracycline responsive element (Tet-RE; Gossen et al., 1992, PNAS 89, 5547-5551). In the absence of Tet, tTA protein binds to the Tet operator sequences and induces a high level expression of the gene of interest. In the presence of Tet, tTA protein binding to the Tet operator, or the Tet-RE, is prevented and thus the transcription of the gene of interest is suppressed. With this method, it is possible to prevent toxicity associated with leakiness of an inducible regulatory region, for example the tet-responsive promoter associated with a mediator, while still allowing regulation by an exogenous agent, for example, tetracycline. By driving expression of tTA with a cell-specific promoter, for example but not limited to TP1, it is possible to achieve ablation of that cell population, and, if required, prevent ablation by the addition of tetracycline. In this embodiment, the expression of a mediator is under the control of a tet-responsive element. When cell-specific tTA expression is activated, then expression of a mediator is accelerated due to the binding of the tet-responsive element by tTA. The expression of the mediator may be reduced, if desired by the addition of tetracycline that prevents tTA binding with Tet-RE.

Background levels of leakiness due to a regulatory region, for example the tet-responsive element, or the GALUAS, associated with a minimal promoter, can be controlled if desired; by co-expressing an inhibitor of mediator activity along with the mediator. For example, in the case of Barnase, Barstar may be coexpressed at low levels to detoxify any Barnase that might be expressed. When the level of Barnase expression is increased, the effect of Bartar is negated. Other mediator-inhibitor combinations may also be used. Such a system is described in Example 5.

Therefore, the present invention also pertains to a method of introducing a direct mediator into a host organism comprising, introducing at least one chimeric construct comprising:

i) an inducible, temporal, or cell specific regulatory region in operative association with a direct mediator; and

ii) an additional regulatory region exhibiting minimal activity and in operative association with an inhibitor, wherein the inhibitor is specific for the direct mediator, and

propagating the host animal.

It is contemplated that the chimeric construct may be introduced within the host on the same vector and at the same time, or the inducible, temporal, or cell specific regulatory region in operative association with a direct mediator described above may be introduced into the host using a separate vector from that used to introduce the additional regulatory region exhibiting minimal activity and in operative association with an inhibitor. In this latter case, it is preferred that the construct comprising the inhibitor is introduced into the host before the construct comprising the direct mediator.

In order to ensure that transgenic animals of the present invention may be propagated so as to produce both male and female offspring which may then be used to produce offspring of a desired sex, the expression of the protein that either directly or indirectly mediator the development of a cell may itself be under transcriptional control. In this manner, the expression of the modifier is repressed until activated in the presence of the transcriptional activator or regulatory protein. One example of a regulatory protein is the GAL4 transcriptional activator that binds and activates genes comprising GAL4 binding sites (GALA upstream activating sequence, or GAL4-UAS), however, other activator/binding site combinations may also be used, including but not limited to the tet (tTA-TRE) system. The GAL4 upstream activating sequence from yeast cells is operable only in the presence of a GAL4 transcriptional activator protein. By crossing animals comprising a chimeric construct containing a tissue specific regulatory element, for example but not limited to TP1, and GAL4-UAS, in operative association with a direct or indirect mediator, selective killing of sperm cells can be produced while still permitting propagation of the transformed animals.

The embodiment of the invention comprising an indirect mediator may necessitate exposing sperm from the animal to a compound that, in the presence of the indirect mediator, produces metabolites that are toxic to the cell. To expose the sperm to the compound, the compound may be administered to the animal prior to mating the animal, or the sperm may be directly exposed to said induction factor by contacting sperm from an animal with the induction factor in vitro. In the case where the indirect modifier is HSV tk, and the indirect modifier gancyclovir, gancyclovir may be administered via any suitable method, for example but not limited to injection, at a dose from about 0.5 mg/km body weight, to about 25 mg/kg body weight. Preferably the dose is from about 5 mg/kg body weight to about 15 mg/kg body weight.

According to another embodiment of the present invention, the sex selection gene may comprises a detectable marker, such as a fluorescent marker, for example green fluorescent protein (GFP). When a selectable marker is present on one of either the X or Y chromosomes, sperm containing the selectable marker can be easily separated in vitro, for example by cell sorting. The transgenic animal having GFP is of interest, because the marker permits sorting of sperm by flow cytometry (FACS) into X and Y bearing populations, and permits histochemical examination of sperm production in the testis and epididymis.

Therefore, the present invention is also directed to a method for the sorting of sperm comprising;

i) introducing in a male animal at least one chimeric construct comprising a regulatory region that is active post-meiotically and in operative association with a sex selection gene, the regulatory region, the sex selection gene, or both the regulatory region and the sex selection gene are bound by nucleotide sequences that target said chimeric construct to one of either a Y or X chromosome; and

ii) separating the sperm according to presence of the detectable marker.

If desired, the animal may be propagated using the separated sperm either with or without the detectable marker, Preferably, the sex selection gene, used in the step of introducing, comprises a detectable marker, for example but not limited to a green fluorescent protein.

In order to target the gene of interest, the at least one sex selection gene, or a combination thereof, to the appropriate X or Y chromosome, targeting means such as homologous recombination can be used. Such a targeting means may comprise flanking the gene to be inserted within the chromosome with nucleotide sequences that are homologous with specific sites localized on either the X or Y chromosome to promote recombination. For example, targeting means may comprise regions of homology with the X chromosome flanking the HPRT locus, or the Y chromosome flanking the SRY locus.

A transgenic male animal formed according to this invention comprises a regulatory region that is active post-meiotically on one of either the Y or X chromosome, and at least one sex selection gene in operative association with the regulatory region which is capable of identifying, modifying or destroying a cell in which it is contained. Progeny of the transgenic male animal formed according to the present invention also fall within the scope of the invention

The technology described herein can easily be applied to any non-human animal, for example, but not limited to agricultural species such as cattle, poultry, swine, sheep, etc., thereby allowing easy transgenic manipulation and selection of the sex of the progeny. Advantageously, offspring from animals manipulated according to the invention can be propagated without cloning. However, if desired the progeny may be clonally propagated in order to maintain the transgene within the desired transgenic animal.

The above description is not intended to limit the claimed invention in any manner, furthermore, the discussed combination of features might not be absolutely necessary for the inventive solution.

The invention will now be described as it relates to particular examples of sex selection in the mouse. The examples describe particular embodiments of the invention, but are not to be construed as limiting. The invention encompasses such modifications to the exemplified embodiments as would occur to one skilled in the art.

### EXAMPLES

The regulator region of the mouse transition protein 1 gene (TP1) is introduced in front of one of three different genes that are either sex selection genes or genes of interest. The three different genes include the thymidine kinase gene from herpes simplex virus (HSVtk), the gene encoding green fluorescent protein (GFP), or the gene encoding the yeast GAL4 transcriptional activator (GAL4), thereby forming the following three transgenes:

(a) TP1-HSV TK;

(b) TP1-GFP;

(c) TP1-GAL4.

A second regulatory region comprising a basal promoter from the thymidine kinase gene, GAL4 UAS, which contains binding sites for the GAL4 transcriptional activator protein, is placed in front of a gene of interest, either a gene encoding Barnase, or the gene encoding GFP to form the following two transgenes:

(d) core-GAL4 UAS-Bamase, and

(e) core-GAL4 UAS-GFP.

The function of the five different transgenes is described below.

The HSV tk protein encoded by transgene (a) is lethal to a cell in the presence of gancyclovir, since the kinase produces toxic metabolites from gancyclovir.

The GFP encoded by transgene (b) and (e) is not lethal to cells, but provides a readily identifiable fluorescent marker.

The GAL4 transcription activator protein (GAL4) encoded by transgene (c) does not have a natural target in the mammalian genome, and will uniquely activate genes that have regulatory regions comprising GAL4 binding sites, such as GAL4 UAS, as encoded by transgenes (d) and (e). When the GAL4 UAS is placed in front of the Barnase or GFP genes, these genes will only be active in the presence of the GAL4 transcriptional activator protein. The Barnase protein encoded by transgene (d) is lethal to a cell because it destroys the RNA in that cell.

Preparation of TP1-GFP

The GFP coding region is amplified from the plasmid pEGFP-C1 (Clontech) by using the following pair of primers:

(SEQ ID NO:1)

5′-GGAATTCGCCACCATGGTGAGCAAGGG-3′ and

(SEQ ID NO:2)

5′-GAAGATCTTTACTTGTACAGCTCGTCCATGC-3′.

The Amplified PCR product is digested by EcoRI and BglII and cloned into EcoRI-BglII-digested pXJ40 (Xiao et al., 1991, Cell 65:551-568) to form pXJ40-GFP. The region including the intron, GFP coding sequence, and SV40 polyA site from the pXJ40-GFP plasmid is amplified using standard techniques known within the art.

The TP1 promoter is amplified according to the sequence information in Yelick et al. (Genomics, 1991;11:687-694) using the following pair of primers and cloned into pBluescript KS using HindIII and XhoI to form pKS-TP1.:

| | |
|---|---|
| 5′-CCGCTCGAGCGCATAAGAGTCCCAAAGCTGC-3′ | (SEQ ID NO:3) |
| and | |
| 5′-CCCAAGCTTGGGTACTTTCTGCCGAAATGAG-3′ | (SEQ ID NO:4) |

The amplified fragment comprising the intron, GFP coding sequence, and SV40 polyA site from the pXJ40-GFP is cloned into pSK-TP1 to form pKS-TP1-GFP.

Preparation of TP1-HSV tk

The HSV tk gene is amplified with primers having HindIII and XhoI sites and cloned into pKS-TP1, to form pKS-TP1-HSVtk.

Preparation of TP1-GAL4

The same primers used for the amplification of the intron, GFP coding sequence, and SV40 polyA site from the pXJ40-GFP are used to amplify the region including the intron, GAL4 coding sequence, and SV40 polyA from the plasmid pXJ40-GAL4. This amplified fragment is cloned into pSK-TP1 (see preparation of TP1-GFP) to form pSK-TP1-GAL4.

Preparation of Core-GAL4 UAS-Barnase

The GAL4 promoter is cloned into pSK to form pSK-GAL4.

The Barnase coding region is amplified from the plasmid pMT416 (Hartley et al. 1988) by the following pair of primers:

(SEQ ID NO:5)

5′-GGAATTCCATGGCACAGGTTATCAACACGTTTG-3′ and

(SEQ ID NO:6)

5′-GAAGATCTTTATCTGATTTTTGTAAAGGTC-3′.

This PCR product is digested by EcoRI and BglII and cloned into EcoRI-BglII-digested pXJ40 (Xiao et al., 1991) to form pXJ40-Bamase.

The same set of primers used to obtain the intron, GFP coding sequence, and SV40 polyA site from the pXJ40-GFP are used to amplify the region including the intron, the Bamase coding sequence, and SV40 polyA from pXJ40-Bamase. This amplified fragment is cloned into pSK-GAL4 to form pSK-GAL4-Barnase.

Preparation of Core-GAL4 UAS-GFP Cloning Strategy

The amplified fragment comprising the intron, GFP coding sequence, and SV40 polyA site from the pXJ40-GFP (see preparation of TP1-GFP) will be cloned into pSK-GAL4 (see core-GAL4 UAS-Barnase) to form pSK-GAL4-GFP.

These trasgenes are introduced into mice via the transformation of embryonal stem cells (Robertson, E. J., 1991,. Biol. Reprod. 44:238-45). Presence of these transgenes in mice is confirmed by Southern blot analysis and PCR.

In Examples 1 to 4, transgenes (a) to (e) are introduced separately into mice. Transgenes (a), (b) and (c) are introduced into embryonal stem cells and are targeted to a transcriptionally active region of either the X or the Y chromosome, depending on which gamete is desirable in producing offspring. If the X chromosome is targeted, exclusively male offspring can be produced. If the Y chromosome is targeted, exclusively female offspring can be produced. This targeting is done using gene-knockout techniques as are known to one of skill in the art. The transgenes are prepared so as to be flanked by sequences that are homologous to either the X or Y chromosome sequences to be targeted, and homologous recombination in stem cells inserts the transgenes into the desired location When targeting the X chromosome, the transgene will be introduced in a region flanking the HPRT locus. Transgenes (d) and (e) may introduced by DNA microinjection, as it is not necessary that these transgenes be located on a sex chromosome.

In Examples 1 to 4, male founder animals will be produced having one of transgenes (a), (b), (d) and (e), respectively. For transgene 3, female founder animals are produced, and are incorporated in Examples 3 and 4. In the Examples, the region of the X chromosome flanking the HPRT locus is targeted which allows destruction of X gametes in an animal bearing transgenes (a), and (d), and which allows fluorescent marking of the X gametes in an animal bearing transgenes (b) and (e).

### Example 1

Killing Sperm Cells Using an Indirect Modifier as a Sex Selection Gene

The mice transformed with transgene (a), as described above, express HSV tk on the X chromosome of maturing sperm.

Gancyclovir. from about 5 to about 15 mg/kg body weight, is administered to transgenic mice expressing HSV tk via injection. Upon gancyclovir administration, HSV tk forms toxic metabolites that leads to the ablation of sperm comprising an X chromosome. The TP1 regulatory region is activated post-meiotically, so that Y bearing sperm are not effected. Thus, the transformed male mouse comprises exclusively Y bearing sperm and produces male offspring, upon gancyclovir administration. An exemplary gancyclovir administration regime comprises two weeks of administration prior to mating, thereby destroying X chromosome-bearing sperm prior to mating.

As an alternative to gancyclovir administration to the animal, from about 5 to about 15 ng/ml of gancyclovir is mixed in with mature sperm in order to kill cells comprising an X chromosome. This incubation period may be in the order of minutes to hours, depending upon the concentration of gancyclovir used and the source of sperm being treated. These sperm are used for in vitro fertilization applications following standard methods known to one of skill in the art. This strategy permits transmission of the transgene to progeny, simply by omitting the administration of gancyclovir, which allows production of the X chromosome having the transgene.

Male mice carrying transgene HSV tk (transgene (a)) are crossed non-transgenic female mice. Without gancyclovir administration, half of the offspring are male and half are female. The female offspring carry the transgene on the X chromosome. After two weeks of gancyclovir administration, male mice carrying transgene (a) are crossed with non-transgenic female mice. The resulting offspring are all male.

### Example 2

Producing Animals with a Marker as a Sex Selection Gene

Mice are transformed with TP1-GFP (construct (b)) as outlined above. Mice transformed with transgene (b) targeted to the X chromosome express GFP in maturing X chromosome containing sperm and result in the fluorescence of these sperm cells. Aside from serving as a control, the fluorescence of GFP also permits sorting of sperm by FACS (fluorescence activated cell sorting; Galbraith, D. W., Anderson, M. T., and Herzenberg, L. A., 1999, Methods Cell Biol 58:315-41; Orfao, A. and Ruiz-Arguelles, A., 1996, Clin Biochem 29:5-9) into X and Y bearing populations.

Sperm is removed from the epididymis of male mice transformed with TP1-GFP and are examined using fluorescence microscopy. It is determined that one half of the cells fluoresce.

### Example 3

Killing Cells Using a Direct Mediator as a Sex Selection Gene.

Male mice transformed with core-GAL4 UAS-Barnase (transgene (d)), as outlined above, are mated to female mice bearing TP1-GAL4 (transgene (c)). Male progeny of this cross acquire TP1-GAL4 from the female parent and CRR-GAL4 UAS-Barnase from the male parent. These male progeny produce GAL4 post-meiotically, since TP1 promotes post-meiotic expression. GAL4 protein activates transgene (d) to produce Barmase. Barnase is lethal to cells, and the X-chromosome bearing sperm of the male progeny are ablated.

When mated to non-transgenic female animal, such male progeny only sire male offspring, since X chromosome bearing sperm are destroyed.

Transgenic mice carrying transgene (c) are mated to mice having transgenes (d). Genetic identification using Southern blot and PCR analysis is used to determine those offspring which carry both transgenes (c) and (d). Animals comprising both transgenes are mated with wild type non-transgenic females. This mating results in exclusively male offspring.

### Example 4

Alternate Method for Producing Animals with a Marker.

Male mice transformed with core-GAL4 UAS-GFP (transgene (e)) are produce as described above and mated to female mice bearing TP1-GAL4 (transgene (c)). Male progeny of this cross acquire transgene (c) from the female parent and transgene (e) from the male parent Thus, GAL4 protein activates transgene (e) to produce GFP. This will result in fluorescence of X-chromosome containing sperm.

Transgenic mice carrying transgene (c) are mated to mice having transgene (e). Genetic identification using Southern blot and PCR analysis is used to determine offspring which carry both transgenes (c) and (e). This offspring produce sperm, characterized in that 50% of the sperm comprise the marker protein GFP, thereby permitting identification of the X-containing sperm.

### Example 5

A Method of Inducing Regulated Cytotoxicity Using the Tetracycline Transactivator System

In this example, a method for suppressing background levels of a cytotoxic agent is described. The Bacillus amyloliquefaciens barnase is a potent ribonuclease (Hartley, 1988, J. Mol. Biol. 202, 913-915) that has been shown to ablate cells. The Bacillus amyloliquefaciens barstar gene binds specifically to barnase, forming a highly stable complex that inhibits barnase activity (Hartlet, 1989 Trends Biochem Sci 14, 450-454; Schreiber and Fersht 1995, J. Mol. Biol. 248, 478-486). As a way of making applications of barnase as a cytotoxin more efficient, a vector, described below, was designed comprising the barstar gene downstream of a minimal promoter and the barnase gene downstream of the tetracycline responsive element (TRE or Tet-RE) associated with a minimal promoter. Therefore, when barnase is expressed at a background level, its toxicity is offset by a similar basal level expression of barstar. In the presence of tetracycline, barnase expression is induced to a high level.

The Tet system employs two chimeric constructs, one expressing a synthetic transactivator protein (tTA) driven by a regulatory element, and the other being a gene of interest under the control of a tetracycline responsive element (Tet-RE; Gossen et al., 1992, PNAS 89, 5547-5551). In the absence of tetracycline, the tTA protein binds to the Tet operator sequences and induces a high level expression of the gene of interest. In the presence of tetracycline, the binding of the tTA protein to the Tet-RE is prevented and thus the transcription of the gene of interest is suppressed.

Plasmids: The barstar gene was amplified from pMT-416 (Hartley, 1988, supra) with the primers:

| | |
|---|---|
| 5′-CGGAATTCCACATGAAAAAAGCAGTCA-3′; | (SEQ ID NO:7) |
| and | |
| 5′-CGGGATCCCGTATTAAGAAAGTATGATG-3′. | (SEQ ID NO 8) |

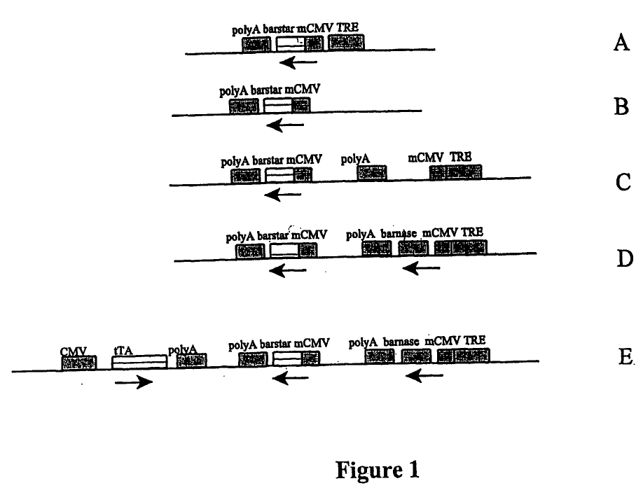

The PCR product was digested with EcoRI and BamHI and inserted into the EcoRI-BamHI sites of pTRE (Clontech, CA), to create pTRE-Barstar (FIG. 1A). This plasmid has the minimal promoter from human cytomegalovirus (CMV), flanked by binding sites for the tetracycline-inducible transactivator protein (tTA).

The PminCMV-Barstar-polyA fragment was amplified with the primers:

| | |
|---|---|
| 5′-TAGGCGTGTACGGTGG-3′; and | (SEQ ID NO 9) |
| 5′-TACCACATTTGTAGAGGTTT-3′, | (SEQ ID NO 10) |

and the fragment blunt-end cloned into the EcoRV site of pBS, to produce pBS-Barstar (FIG. 1B).

To generate pBS-Barstar-TRE-PolyA (FIG. 1C), the XhoI and HindIII fragment containing TRE-PminCMV-SV40polyA was isolated from pTRE and into the XbaI and PstI sites of pBS-Barstar (FIG. 1B). The non-cohesive ends were made blunt with T4 DNA polymerase prior to ligation. The Barnase gene was amplified from pMT416 using the primers:

(SEQ ID NO: 11)

5′-GCTCTAGAGCATGGCACAGGTTATCAACACGTT-3′; and

(SEQ ID NO 12)

5′-GCTCTAGACGTTATCTGATTTTTGTAAAGG-3′.

The PCR product was digested with XbaI and ligated into the XbaI site of pBS-Barstar-TRE-PolyA, resulting in pBS-Barstar-Bamase (FIG. 1D). The XhoI and HindIII fragment from pTet-Off (Clontech, CA) containing CMV-tTA was inserted into the XhoI and HindIII sites of pBS-Barstar-Bamase to form pBS-Tet-off-Barstar-Barnase (FIG. 1E).

The minimal CMV promoter and Luc reporter gene was amplified from pTRE-Luc (Clontech, CA) using a forward primer:

| | | |
|---|---|---|
| | 5′-CCGCTCGAGTAGGCGTGTACGG-3′; and a | (SEQ ID NO 13) |
| reverse primer: | 5′-TCCCCGCGGTTACAATTTGGACTTTCCGC-3′. | (SEQ ID NO 14) |

The PCR product was digested with XhoI and SacII and ligated into the XhoI and SacII sites of pTRE to generate pPminCMV-Luc. To produce pCMV-Luc, which expresses luciferase constitutively, the luciferase gene was amplified and cloned into pXJ40 (Xiao et al., 1991, Cell 65, 551-568). pCMV-GFP was a product from Clontech.

Cell culture and transient transfection: Human 293 cells from embryonal kidney were maintained at 37° C., 5% CO₂ in α-MEM supplemented with 10% fetal bovine serum. Transfections by calcium phosphate co-precipitation were performed according to standard protocols. Transfections were carried out in 35 mm dishes, using 1 μg of plasmid together with 0.5 μg of pCMV-Luc or pCMV-GFP. Control trasfections contained 1 μg of empty pTRE plasmid DNA together with 0.5 μg of either pCMV-Luc or pCMV-GFP. Tetracycline was used at a concentration of 2 μg/ml, and added shortly after cells were transfected, unless otherwise indicated. All data were from three replicate plates and the experiments were repeated twice.

Luciferase activity: Twenty-four hours following transfection, media was removed, cells were washed with PBS and luciferase assays were performed as described in Yin et al (1996) with Promega luciferase assay system, using 10 μl lysate and 50 μl luciferase substrate. Activity was measured with a Turner TD-20e luminometer. Protein concentrations in different lysates were determined by Bradford assays (Bio-Rad), and luciferase activity per μg protein determined.

a) Barstar Driven by the Minimal CMV Promoter is Enough to Inhibit the Basal Level of Barnase Activity but not to Rescue Cells when Barnase was Activated by tTA

The luciferase reporter gene was placed downstream of the minimal CMV promoter lacking tTA binding sites to create pPminCMV-Luc, and its activity was compared to that of pTRE-Luc. Cells were transfected with either plasmid, and both plasmids produced similar levels of luciferase activity.

When these two plasmids were cotransfected with pTet-Off, which makes the transactivator protein, the luciferase expression of pPminCMV-Luc did not change in response to tetracycline (as it does not comprise a Tet-RE) while that of pTPE-Luc was strongly induced, confirming that the transactivator was functional in human cells.

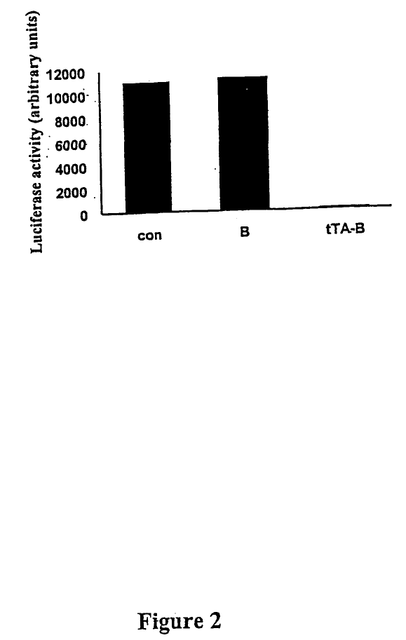

Human cells transfected with pCMV-Luc either with or without pBS-Barnase-Barstar, exhibited similar luciferase activity (FIG. 2), indicating that the amount of barnase being produced from the pBS-Barstar-Barnase plasmid was at a sub lethal level. Without wishing to be bound by theory, the presence of a basal levels of barstar inhibited any barnase that was being made. A plasmid carrying barnase under control of the pTRE promoter could not be prepared without co-producing barstar in E. coli. Once prepared the construct was tested in human (293) cells. The transformed cells all died and no luciferase activity was detected, even in the absence of the transactivator. This plasmid made no barstar in 293 cells, indicating that basal levels of barnase expressed from the pTRE promoter are sufficient to kill cells.

The gene encoding the transactivator protein was included into the plasmid carrying barstar-barnase to produce pBS-CMV-tTA-Barnase-Barstar (FIG. 1E). Transfection of pBS-CMV-tTA-Bamase-Barstar into 293 cells yielded little luciferase activity compared to control transfections (FIG. 2), indicating that induced bamase production was killing the transfected cells. To confirm that the lethal effect was due to the induction of barnase by tTA, the CMV promoter driving expression of tTA was replaced with the tissue specific promoter TP1 (Yelick et al., Genomics, 1991;11:687-694), which is not active in 293 cells. Cells transfected with this plasmid produced high levels of luciferase, similar to the control sample.

These results demonstrate that basal levels of barstar driven by the minimal CMV promoter were sufficient to inhibit the basal level of barnase activity, but not sufficient to rescue cells when barnase was activated by tTA.

b) Cytotoxic Effect of Barnase was Regulated by Tetracycline

To test if the lethal effect exerted by barnase was regulated by Tetrecycline, cells were split onto two plates, 12 hr after co-transfection with pBS-CMV-tTA-Barnase-Barstar and pCMV-GFP; one plate was treated with tetracycline. It was observed that transfection with pBS-CMV-tTA-Barnase-Barstar resulted in loss of GFP in both plates in the presence or absence of tetracycline. GFP was readily visible in control samples that had been co-transfected with pTRE (with no insert) and pCMV-GFP. These results demonstrate that transfection with pBS-CMV-tTA-Barnase-Barstar impaired the transfected cells very quickly.

Pretreating cells with tetracycline for 24 hours prior to co-transfection with pBS-CMV-tTA-Barnase-Barstar and pCMV-GFP resulted in the production of GFP positive cells. Cells that were not treated with tetracycline and that were co-transfected with the same constructs made little or no GFP. To further evaluate the effect of a tetracycline pretreatment, cells were either pretreated, or not pretreated, with tetracycline and co-transfected with pBS-CMV-tTA-Barnase-Barstar and pCMV-Luc.

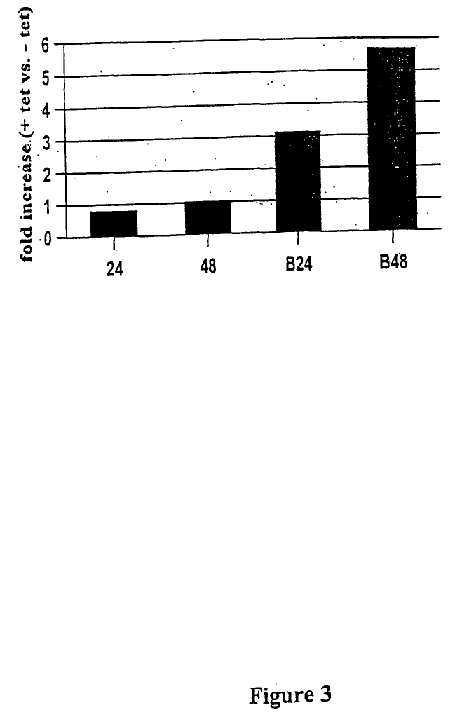

As shown in FIG. 3, 24 hours after transfection there was about three times more luciferase activity in the cells treated with tetracycline than those not treated, and after 48 hours the difference became almost 6-fold. The luciferase activity for cells co-transfected with pTRE and pCMV-Luc remained constant over this time period. The results demonstrate that the lethal effect of barnase can be prevented by suppressing its expression with tetracycline.

### Example 6

Targeted GFP Expression in Trangenic Mice Testis.

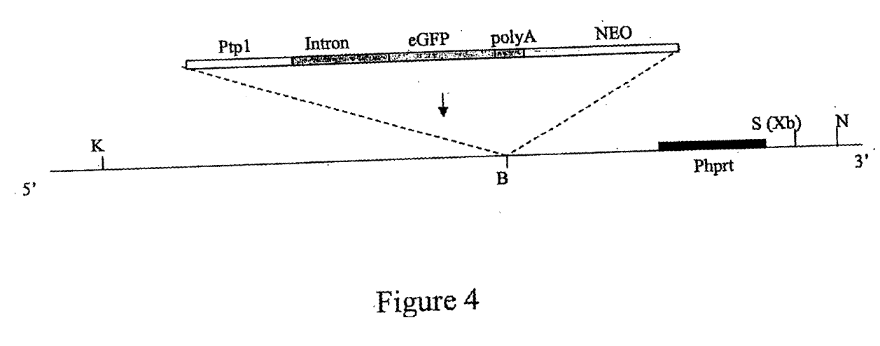

The targeting vector graphically illustrated in FIG. 4 was employed in an aspect of an embodiment of the present invention to target GFP expression in transgenic mice testis. HPRT flanking region was mapped by 8 restriction enzymes, including: B (BamHI), E (EcoRI), H (HindIII), K (KpnI), P (PstI), S (SacI), Xb (XbaI) and Xh (XhoI). The genomic fragment containing the promoter of Hprt (Phprt) was digested by KpnI and SacI and cloned into KpnI-XbaI digested pBluescript. The non-cohesive ends were made blunt prior to ligation. BamHI site on the genomic fragment was chosen to insert the fragment containing TP1 promoter (Ptp1), an intron, enhanced GFP coding sequence (eGFP), polyA and a NEO expression cassette. The left homologous arm from KpnI to BamHI is around 5 kb and the right homologous arm from BamHI to SacI is about 3 kb. The targeting vector was linearized by NotI (N) before electroporation into ES cells.

GFP is Transcribed in Transgenic Mice Testis

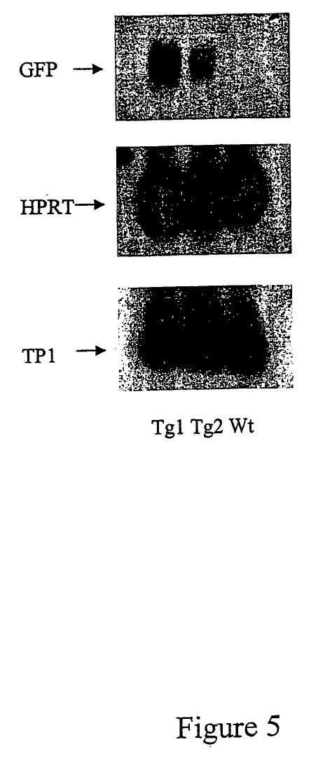

RNA was extracted from about 8 week old wild type mouse testis (Wt) and two transgenic mice testis (Tg1 and Tg2). 10 μg total RNA was loaded on each lane. GFP, HPRT and TP1 coding regions were used as probes for each blot. The results are shown in FIG. 5. The results indicate that GFP may be efficiently targeted and transcribed in transgenic mouse testis.

GFP is Detected Only in Transgenic Mice Testis

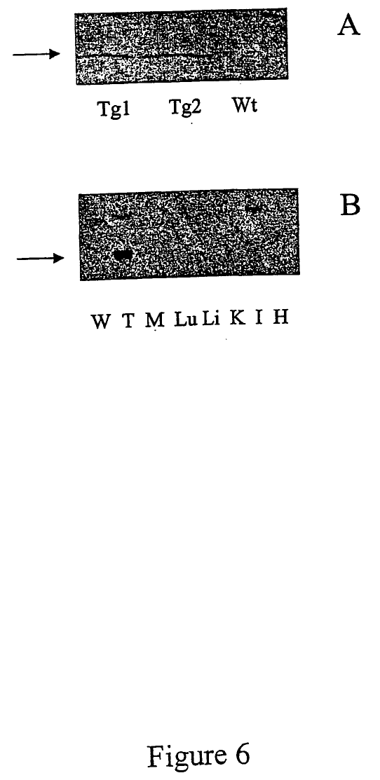

Proteins were extracted from about 8 week old wild type mouse testis (Wt) and two transgenic mice testis (Tg1 and Tg2). 60 μg protein was loaded on each lane and separated on 12% acrylamide gel. GFP(FL) from Santa Cruz Biotechnology Inc. was used as primary antibody and anti-rabbit IgG-AP as secondary antibody in Western blots. The results are shown in FIG. 6. The arrow shown in FIG. 6 indicates the expected size for GFP. As shown in FIG. 6B, proteins were extracted from heart (H), intestines (I), kidney (K), liver (Li), lung (Lu), muscle (M) and testis (T) of a transgenic mouse. Protein from wild type mouse testis (W) was used as negative control. Western blot was performed as described above. The results indicate that GFP protein is detected only in transgenic mice testis.

All publications cited herein are incorporated by reference.

Various modifications may be made without departing from the invention. It is understood that the invention has been disclosed herein in connection with certain examples and embodiments. However, such changes, modifications or equivalents as can be used by those skilled in the art are intended to be included. Accordingly, the disclosure is to be construed as exemplary, rather than limiting, and such changes within the principles of the invention as are obvious to one skilled in the art are intended to be included within the scope of the claims.

### References

Ellis et al. 1988. Sex determination of bovine embryos using male-specific DNA probes. Theriogenology 29:242.

Hartley, R. W. 1988. J.Mol. Biol. Vol. 202, p.913-915.

Johnson, L. A. 1997. Advances in gender preselection in swine. J Reprod Fertil Suppl;52:255-66.

Kim, Y., Kremling, H., Tessmann, D. and Engel, W. 1992. Nucleotide sequence and exon-intron structure of the bovine transition protein 1 gene. DNA Seq. 3:123-125.

Yelick, P. C., Kozak, C., Kwon, Y. K, Seldin, M. F. and Hecht, N. B. 1991. The mouse transition protein 1 gene contains a B1 repetitive element and is located on chromosome 1. Genomics 11:687-94.

The embodiments of the invention in which an exclusive property of privilege is claimed are defied as follows:

1. A method for sex selection comprising, introducing a post-meiotically expressed sex selection gene into one of either a Y or X chromosome of a male non-human animal, and propagating said animal.

2. A method for sex selection comprising, introducing in a male animal at least one chimeric construct comprising a regulatory region that is active post-meiotically and in operative association with a sex selection gene, wherein said regulatory region, said sex selection gene, or both said regulatory region and said sex selection gene are bound by nucleotide sequences that target said chimeric construct to one of either a Y or X chromosome and propagating said non-human animal.

3. The method according to claim 2 wherein said step of introducing comprises transgenic manipulation.

4. The method according to claim 2 wherein said step of introducing comprises donation from a parent bearing said chimeric construct.

5. The method according to claim 2, wherein said post-meiotic regulatory element, used in said step of introducing, is a transition protein 1 (T1) regulatory region.

6. The method according to claim 5, wherein said sex selection gene, used in said step of introducing, encodes a direct mediator.

7. The method according to claim 6, wherein said direct mediator comprises a ribonuclease.

8. The method according to claim 6, wherein said ribonuclease is Barnase.

9. The method according to claim 5, wherein said sex selection gene, used in said step of introducing, encodes an indirect mediator.

10. The method according to claim 9 further comprising a step of adding a compound to at least one sperm cell expressing said indirect mediator, wherein said indirect mediator modifies cell development in the presence of said compound.

11. The method according to claim 9, wherein said step of adding involves exposing isolated sperm to said compound.

12. The method according to claim 9, wherein said step of adding involves administering said compound to said male non-human animal.

13. The method according to claim 10, wherein said indirect mediator comprises thymidine kinase from herpes simplex virus, and said compound comprises gancyclovir.

14. The method according to claim 5, wherein said sex selection gene, used in said step of introducing, is in operative association with an inducible regulatory element, and said post-meiotic promoter sequence is in operative association with a gene of interest encoding a regulatory protein capable of activating said inducible regulatory element.

15. The method according to claim 14, wherein said inducible regulatory element comprises a GAL4 upstream activating sequence, and said regulatory protein is a GAL4 transcription activator protein.

16. The method according to claim 2, wherein said sex selection gene, used in said step of introducing, comprises a detectable marker.

17. The method of claim 16 further comprising a step of separating sperm according to presence of the detectable marker prior to propagation, and wherein said animal is propagated using sperm either with or without said detectable marker.

18. The method according to claim 16, wherein said detectable marker is green fluorescent protein.

19. The method according to claim 2, wherein said nucleotide sequences that target said chimeric construct, used with said step of introducing, comprise regions of homology with one of the X or Y chromosome to promote recombination.

20. The method according to claim 19, wherein said nucleotide sequences comprises sequences of homology within the X chromosome flanking the HPRT locus.

21. The method according to claim 14, wherein said sex selection gene, used in said step of introducing, is in operative association with an inducible regulatory element, and is introduced into a first non-human animal, said method further comprising a second introducing step, comprising introducing into a second nonhuman animal said post-meiotic promoter sequence is in operative association with a gene of interest encoding a regulatory protein capable of activating said inducible regulatory element, and mating said first and second non-human animals to produce progeny.

22. The method according to claim 21, wherein said inducible regulatory element comprises a GAL4 upstream activating sequence, and said regulatory protein is a GAL4 transcription activator protein.

23. A chimeric construct comprising a post-meiotically active regulatory region in operative association with a sex selection gene, both of said regulatory region and said sex selection gene bound by nucleotide sequences that target said chimeric construct to one of either a Y or X chromosome.

24. A transgenic non-human male animal comprising the chimeric construct of claim 23.

25. The transgenic non-human male animal according to claim 24, wherein said post-meiotically active regulatory region comprises a transition protein 1 promoter.

26. The transgenic male non-human animal according to claim 24 wherein the sex selection gene encodes a direct mediator, an indirect mediator, or a marker.

27. Progeny of the transgenic male non-human animal of claim 24.

28. A pair of chimeric constructs comprising a first and a second chimeric construct, said first chimeric construct comprising:
a first regulatory region in operative association with a gene of interest encoding a regulatory protein,
said second construct comprising:
a second regulatory region and an inducible regulatory element capable of regulating the activity of said regulatory region in the presence of said regulatory protein, in operative association with a sex selection gene,
wherein said first, said second, or both said first and said second chimeric constructs are bound by nucleotide sequences that target said first, said second, or independently both said first and said second construct, to one of either a Y or X chromosome; and wherein said first, said second, or both said first and said second regulatory region is a post-meiotically active regulatory region

29. The pair of chimeric constructs according to claim 28, wherein said post-meiotically active regulatory region is a transition protein 1 promoter.

30. The pair of chimeric constructs according to claim 29 wherein, said regulatory protein is GAL4, and said inducible regulatory element is a GAL4-UAS

31. The pair of chimeric constructs of claim 29, wherein said nucleotide sequences are HPRT nucleotide sequences and target said first, said second, or independently both said first and second chimeric construct, to the X chromosome.

32. The pair of chimeric constructs of claim 29, wherein said nucleotide sequences are SRY nucleotide sequences and target said first, said second, or independently both said first and second chimeric construct, to the Y chromosome.

33. A transgenic non-human male animal comprising said first, said second or said pair, of chimeric constructs of claim 28.

34. The transgenic male non-human animal according to claim 33, wherein the sex selection gene encodes a direct mediator, an indirect mediator, or a marker.

35. Progeny of the transgenic male non-human animal of claim 34.

36. A method for sex selection comprising, introducing said pair of chimeric constructs of claim 28 into a male non-human animal, and propagating said animal.

37. A method for sex selection comprising, introducing said pair of chimeric constructs of claim 29 into a male non-human animal, and propagating said animal.

38. A method for sex selection comprising, introducing said pair of chimeric constructs of claim 30 into a male non-human animal, and propagating said animal.

39. A method for sex selection comprising, introducing said pair of chimeric constructs of claim 31 into a male non-human animal, and propagating said animal.

40. A method for sex selection comprising, introducing said pair of chimeric constructs of claim 32 into a male non-human animal, and propagating said animal.

41. The method according to claim 14, wherein said inducible regulatory element comprises a tet-responsive element, and said regulatory protein is a tet-trnsactivator protein.

42. The method of claim 6, wherein in said step of introducing, said chimeric construct further comprises an additional regulatory element in operative association with a gene encoding an inhibitor, said inhibitor being specific for said direct mediator.

43. The method of claim 42, wherein said direct mediator is barnase, and said inhibitor is barstar.

44. A method of introducing a direct mediator into a host organism comprising, introducing at least one chimeric construct comprising:
i) an inducible, temporal, or cell specific regulatory region in operative association with a direct mediator; and
ii) an additional regulatory region exhibiting minimal activity and in operative association with an inhibitor, said inhibitor being specific for said direct mediator, and
propagating said host animal.

45. The method of claim 44, wherein said direct mediator is bamase, and said inhibitor is barstar.
